(19) United States
(12) Patent Application Publication
(10) Pub. No.: US 2004/0204647 A1
(43) Pub. Date: Oct. 14, 2004

(54) Medical implant system

(75) Inventors: Thomas Grupp, Donzdorf (DE); Josef Kozak, Tuttlingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/765,724

(22) Filed: Jan. 26, 2004

Related U.S. Application Data
(63) Continuation of application No. PCT/EP02/07927, filed on Jul. 17, 2002.

(30) Foreign Application Priority Data
Jul. 28, 2001 (DE) 101 37 011.3

(51) Int. Cl. A61B 6/00

(52) U.S. Cl. 600/431; 606/102

(57) ABSTRACT

In the case of a medical implant system with an implant made of a composite material in which glass fibers are embedded, to obtain information on physical states of the implant in its environment it is proposed that a sensor element which is embedded in the implant and comprises at least one of the glass fibers is coupled to a measuring device which determines a physical property of the sensor element or its environment and changing of this property.

The present disclosure relates to the subject matter disclosed in International application No. PCT/EP02/07927 of Jul. 17, 2002, which is incorporated herein by reference in its entirety and for all purposes. International application No. PCT/EP02/07927 claims the benefit of German patent application no. 101 37 011.3 filed on Jul. 28, 2001.

### BACKGROUND OF THE INVENTION

The invention relates to a medical implant system with an implant made of a composite material in which glass fibers are embedded.

Medical implants, for example bone plates, intramedullary nails, endoprostheses, osteosynthesis systems for the spinal column, etc., are usually produced from metal materials, but there are also known implants which consist of a composite material in which glass fibers are embedded for reinforcement; in particular, such medical implants consist of selected sterilizable plastics, such as polyether ether ketone, polyamides, etc.

When these implants are in situ in the body, they are subjected to various influences, for example various stresses and strains, temperature developments or chemical environments. It would be of interest to the treatment procedure to find out about these different parameters, since they provide information on how healing progresses or on problems possibly occurring.

It is an object of the invention to improve an medical implant system of the generic type in such a way that information on physical properties in the implant and in its environment can be obtained.

### SUMMARY OF THE INVENTION

In the case of a medical implant of the type described at the beginning, this object is achieved according to the invention by a sensor element which is embedded in the implant and comprises at least one of the glass fibers being coupled to a measuring device which determines a physical property of the sensor element or its environment and changing of this property.

Consequently, at least one glass fiber embedded in the composite material of the implant is used for the transmission of signals which provide information on the physical properties of the implant or the environment of the implant.

In this case, the term “glass fiber” is understood as meaning all fibrous substances which can be embedded in the composite material and are capable of carrying and transmitting electromagnetic radiation; these fibers preferably consist of quartz glass, but other substances may also be used, for example synthetic fibers, known as Plastic Optical Fibers (POFs).

It is advantageous if the glass fibers are embedded in the composite material as mechanical reinforcement.

In particular, it may be provided in this case that the glass fibers are disposed in the form of a woven fabric, a knitted fabric or a fleece, that is to say form a network which is embedded as a whole in the composite material and reinforces the latter as a result.

Depending on the mechanical requirements, the glass fibers may in this case be concentrated in specific regions of the implant, or else be distributed over the entire extent of the implant.

The measuring device is preferably formed in such a way that it feeds electromagnetic radiation into the sensor element and determines physical properties of the sensor element or of its environment from the type of radiation that passes through and/or is reflected.

According to a preferred embodiment, the glass fiber of the sensor element is provided with a radiation-reflecting coating.

In the case of a first preferred embodiment, the sensor element substantially consists of the glass fiber forming a sensor fiber. In the case of this embodiment, the glass fiber embedded in the composite material is consequently at the same time the sensor and transmission element for the electromagnetic radiation.

Quite a large number of different configurations in which the glass fiber acts as a sensor fiber are possible, for example at least one region acting as a Bragg grating may be incorporated in the sensor fiber. In such a region, which has periodic changes of the refractive index in the longitudinal direction of the sensor fiber, radiation is reflected, said radiation being superposed during the reflection and only intensifying in the return direction for quite specific wavelengths. This wavelength depends on the periodicity of the Bragg grating region and changes with this periodicity. Any change in length of the sensor fiber or any change in the periodicity of the Bragg grating that occurs on account of external influences can in this way be detected in the form of a wavelength shift.

In the case of another preferred embodiment, it may be provided that a substance that is induced to fluoresce by the fed-in electromagnetic radiation and the fluorescent properties of which undergo changes under the effect of the environment outside the sensor fiber is embedded in the sensor fiber. These changes may be mechanical changes, but the fluorescent property of the embedded substance can in particular be influenced by the chemical environment of the sensor fiber, for example the fluorescence can be extinguished by certain substances in the environment.

In the case of a further preferred embodiment, it is provided that the radiation-reflecting coating consists of a substance which changes the reflection behavior for the electromagnetic radiation in the sensor fiber under the effect of the environment outside the sensor fiber. As a result, the amount of radiation that passes through and is reflected by the sensor fiber changes, and this can be detected.

Every change of the properties in the radiation can be detected; this may comprise changes of the wavelength, of the phase position, of the polarization, etc., but all that is important is that these changes are in a clearly perceivable relationship with changes of the properties in the environment of the sensor fiber, that is to say for example with changes of the mechanical stress, the temperature or the material composition.

In the case of a further preferred embodiment, it may be provided that the sensor element comprises the glass fiber and a further sensor member which is coupled to the measuring device via the glass fiber. In the case of this configuration, the glass fiber acts substantially as a transmission element between the sensor member and the measuring device.

For example, the sensor member may be a pressure sensor with a flexible membrane and a mirror element which can be moved by the latter and reflects the electromagnetic radiation fed into the glass fiber differently according to position.

In the case of a further embodiment, the sensor member may be a Fabry-Pérot interferometer.

For example, it may in this case be provided that the Fabry-Pérot interferometer is formed as a thin-film interferometer that is brought into contact with the end of the glass fiber and the active film of which undergoes dimensional changes under the influence of the environment. Such an active film may, for example, be in a porous form and swell when it comes into contact with a liquid; in this way it is possible for example to detect whether an implant is still sealed or has a desired or undesired opening with respect to the environment.

In the case of another embodiment, it is provided that the Fabry-Pérot interferometer comprises two glass fibers with polished end faces, the spacing between which can be changed by environmental influences. This configuration is advantageous in particular whenever strains or displacements within an implant are to be detected.

The glass fiber of the sensor element may be connected directly to the measuring device, it being possible for the measuring device to be carried inside the body, but also outside it. In the latter case, the glass fiber is led out from the implant through the body tissue, so that a connection to the measuring device can be established there.

It is particularly advantageous if the measuring device is a microcontroller that is capable of being implanted in the body.

In the case of a particularly preferred embodiment, the glass fiber is connected to a transducer, which exchanges signals with the measuring device without a physical connection.

This transducer may in particular be capable of being implanted in the body, for example it may be a transponder.

In the case of a particularly advantageous embodiment, the transducer is a light source which has an associated light receiver. It has been found that light of different wavelengths can penetrate body tissue to a certain extent, with the result that a transmission of radiation energy is possible by light between a light receiver and a light source, of which part is disposed in the body and part outside it, in particular whenever the light source emits electromagnetic radiation in the range between 650 and 1000 nm.

In the case of a particularly preferred embodiment, the measuring device has an associated radiation transmitter, which transports radiation into the interior of the implant via a glass fiber in the implant. In addition to determining the physical properties of the implant by the coupled-in radiation, such a radiation transmitter can be used for acting on the implant and changing it, for example by heating it up in specific regions or the like.

It may in this case be provided that the transport of the radiation takes place via a glass fiber which is embedded in the implant in addition to the glass fiber of a sensor element, but it may also be provided that the transport of the radiation takes place via the glass fiber of a sensor element. In this case, it is advantageous to use appropriate switching elements that selectively connect the glass fiber to the measuring device and to the radiation transmitter.

Particularly advantageous is a configuration in which the wavelength and intensity of the transported radiation are chosen such that the radiation induces mechanical and/or material changes in the composite material of the implant. For example, it is possible in this way to perform additional hardening of a polymeric composite material in specific regions or, conversely, weakening by destroying the composite material, with the result that the mechanical properties of the implant can be changed in this way in relatively large areas or else locally.

In the case of a particularly preferred embodiment, it is provided in this case that the measuring device and the radiation transmitter have an associated controller, which activates the radiation transmitter in dependence on the measured variables of the measuring device. In the case of this configuration, it is possible to determine the physical data of the implant continuously, for example the mechanical stresses transferred to the implant, which are for example a measure of the healing process; these stresses decrease with increasing stability at the bone connection, since a part of the loads are taken over by the bone. It is then advantageous to reduce the strength of the implant in a way corresponding to this regeneration of the bone connection, with the result that the force-transfer function is increasingly taken over by the healing bone.

The following description of preferred embodiments of the invention serves for a more detailed explanation in conjunction with the drawing, in which:

### BRIEF DESCRIPTION OF THE DRAWINGS

FIG. 1 shows a schematic view of an implant in the form of a bone plate with a wireless connection to a measuring device;

FIG. 2 shows a schematic view of an implant in the form of a plate with a glass fiber reinforcement in the form of a network;

FIG. 3 shows a schematic view of an implant in the form of a bone plate with a measuring device connected to a number of glass fibers and with a radiation source for the introduction of radiation into a glass fiber that is not connected to the measuring device;

FIG. 4 shows a view similar to FIG. 3 with a switching device for the selective connection of glass fibers in the implant to the measuring device or to the radiation source;

FIG. 5 shows a schematic side view of a glass fiber with Bragg grating regions of different periodicity;

FIG. 6 shows a schematic side view of a glass fiber with embedded fluorescent dye particles;

FIG. 7 shows a schematic side view of a glass fiber with a sheathing with changing transmission properties;

FIG. 8 shows a schematic side view of a Fabry-Pérot interferometer connected to a glass fiber, with two pieces of glass fiber that are moved towards each other;

FIG. 9 shows a view similar to FIG. 8 with a dimensionally-changing active film, and

FIG. 10 shows a schematic side view of a glass fiber with a membrane pressure sensor.

### DETAILED DESCRIPTION OF THE INVENTION

The invention is explained below on the basis of the example of a bone plate; however, it is to be understood that the invention can be used generally for medical implants that can be inserted in the body and is not restricted to bone plates.

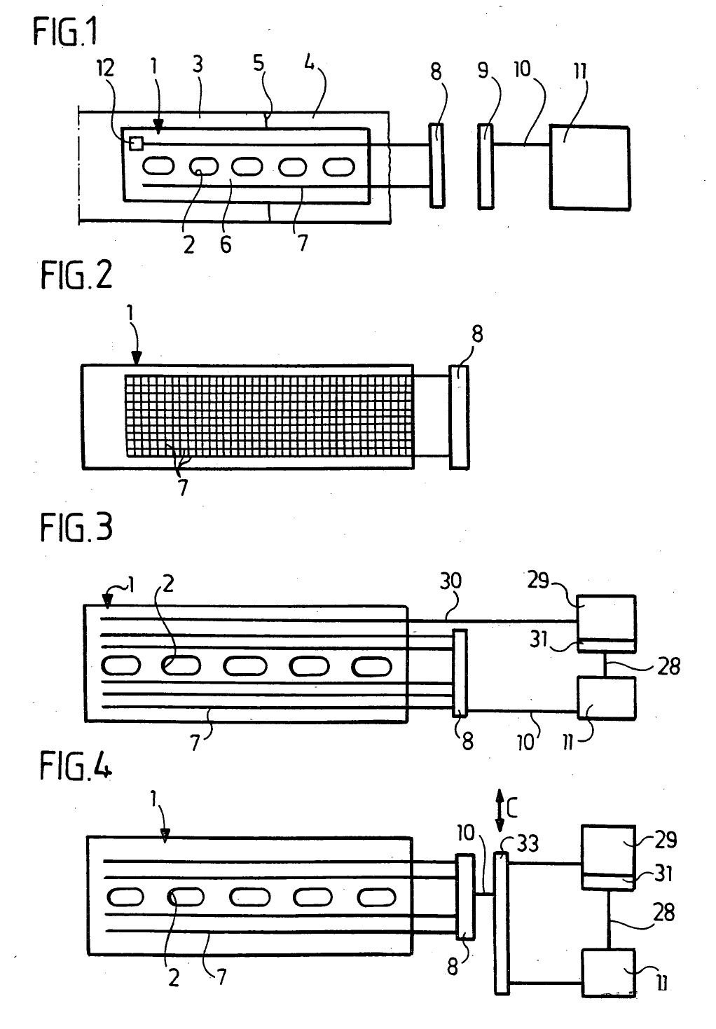

An implant 1 in the form of a bone plate with openings 2 for receiving bone screws is connected in a way known per se by means of bone screws to two bone fragments 3, 4 in such a way that the latter are fixed in a specific relative position with respect to each other, with the result for example that a fracture 5 can heal (FIG. 1). The implant 1 consists of a synthetic material, for example a resorbable plastic such as polylactide (PLLA PL DLLA), polyglycolide (PGA) or trimethylene carbonate (TMC), and glass fibers 7 are embedded in this synthetic material 6. In the exemplary embodiment of FIG. 1 only two individual glass fibers 7 are schematically represented, extending in the longitudinal direction of the plate-shaped implant 1; in the exemplary emdodiment of FIG. 2, a multiplicity of glass fibers 7 are indicated in the form of a network, which is embedded as a whole in the synthetic material 6; the widest variety of arrangements and concentrations of glass fibers in the synthetic material 6 are possible here. The glass fibers reinforce the synthetic material 6 by this embedding, and different distributions in the implant are accordingly chosen, depending on the mechanical strength requirements.

The glass fibers 7 in the exemplary embodiment of FIG. 1 are connected to a transmission element 8, for example a customary transponder, which may be disposed on the implant 1 itself or remote from the implant 1 in the interior of the patient's body or else on the surface of the patient's body; it may in this case also be an optical element, which can receive and emit light, for example a small parabolic mirror, a lens or the like. In the exemplary embodiment of FIG. 1, all the glass fibers 7 disposed in the implant 1 are connected to the transmission element 8; in the exemplary embodiment of FIG. 2, only some of the glass fibers are connected, while others serve exclusively for reinforcing the implant 1. This can be chosen differently from case to case; in the extreme case, it is sufficient to connect a single glass fiber 7 in the implant 1 to such a transmission element 8.

The transmission element 8 has a corresponding associated transmission element 9, which is connected to the measuring device 11 via a line 10. Signals can be exchanged between the transmission elements 8 and 9; these may be electrical signals, optical signals or mechanical signals (ultrasound); all that is important is that electromagnetic energy is transmitted from the transmission element 8 into the glass fiber and, if appropriate, from the glass fiber into the transmission element 8 and is converted in the transmission element 8 into signals which can then be passed in any desired way to the transmission element 9, and consequently to the measuring device 11. If the transmission element 8 is disposed in the interior of the body, the transmission elements 8 and 9 can exchange in particular an electromagnetic radiation with a wavelength of between 650 and 1000 nanometers; this electromagnetic radiation can penetrate the body tissue to a certain depth and can consequently establish a signal connection between the two transmission elements 8 and 9, to be precise both in the inward radiating direction and in the outward radiating direction.

The radiation coupled into the glass fiber 7 in this way is carried in the glass fiber 7 and changed by the latter itself or by a sensor member 12 connected to it, to be precise in a way dependent on the data relating to the physical state of the glass fiber 7, the sensor member 12 or the environment of the same. The radiation then sent in the return direction from the glass fiber 7 to the transmission element 8 is correspondingly changed, and this change can be detected by the measuring device 11, which consequently receives feedback on changes of the physical state of the glass fiber, of the sensor member 12 and/or of the environment of the same.

The possibilities for affecting the electromagnetic radiation fed into the glass fiber 7 are many and varied; changes in length, deformations, mechanical tensile stresses, forces, vibrations, pressures, angles of rotation, electric or magnetic field strengths, currents, temperatures, moisture, ionizing radiations or the concentration or presence of chemical substances can be determined in this way; this is just a selection of the possible physical states that can be detected in this way. Some examples of the influencing of the electromagnetic radiation in a glass fiber are discussed below on the basis of FIGS. 5 to 10.

In FIG. 5, a detail of a glass fiber 7 is represented; provided in this glass fiber are various regions 13, 14, 15, which are spaced apart from one another in the longitudinal direction and in which periodic changes of the refractive index occur in the longitudinal direction of the fiber. These can be produced for example by irradiating a quartz glass fiber, doped for example with germanium dioxide, with ultraviolet light of 240 nm wavelength through a microlithographic mask. This produces in each region 13, 14, 15 an arrangement of a Bragg grating, the periodicity, and consequently the grating constant, being chosen differently in different regions 13, 14, 15.

At each of these Bragg gratings, a quite specific wavelength is reflected by interference radiation; this wavelength is dependent on the periodicity of the grating, and consequently also changes when the latter changes periodicity. Such changing of the periodicity or grating constant may take place due to outside influences, for example strain of the glass fiber, bending of the glass fiber, heating, etc. Since only radiation of a specific wavelength is reflected in each region 13, 14, 15, it is possible to ascertain immediately from the wavelength of the reflected radiation at which region a reflection has taken place; furthermore, the shift of the wavelength provides information on changes of the grating spacings in these regions, that is to say for example information on the strain of the glass fiber in specific regions. This may be different in the regions 13, 14, 15; the measuring device can provide indications on the basis of the reflected radiation as to how great a strain in each of the regions 13, 14, 15 is. Consequently, in particular when a number of such glass fibers are used, exact information about the deformation of the implant 1 in the body is obtained, and thus for example about the progress of healing on the growing together of bone fragments. The strain caused by the forces exerted will be greatest when the bone fragments have not yet grown together, and it will keep decreasing as the healing progresses.

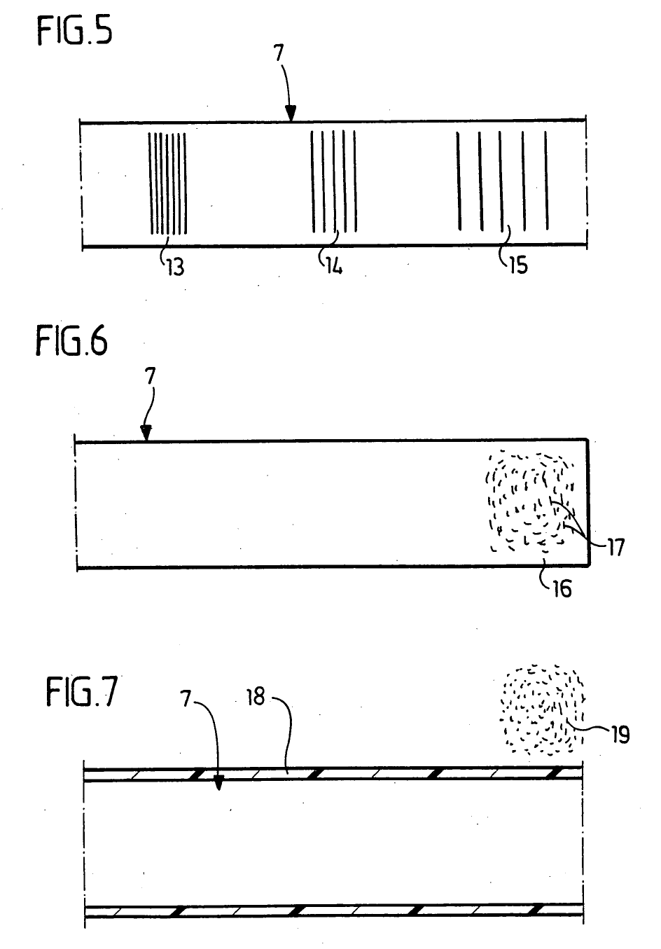

In the case of the exemplary embodiment of FIG. 6, embedded in the glass fiber 7 in a specific region 16 are dye particles 17, which are induced to fluoresce by electromagnetic radiation entering the glass fiber 7. The radiation emitted in this way can be determined by the measuring device. Environmental influences, for example certain chemical substances in the environment of the region 16, can influence the fluorescence, for example the intensity of the fluorescence may be reduced or else the fluorescence extinguished entirely.

In this way, the measuring device receives information on the presence of certain chemical substances in the environment of the region 16. In the case of the exemplary embodiment of FIG. 7, the glass fiber 7 is enclosed with a coating 18, which prevents the electromagnetic radiation carried by the glass fiber 7 from emerging. This coating may react with chemical substances 19 in the environment and thereby undergo such a transformation that the emerging properties of the electromagnetic radiation are changed in the region in which the chemical substance 19 is located, and in this way a change of the reflected radiation is again obtained in dependence on certain chemical substances 19 in the environment of the glass fiber 7.

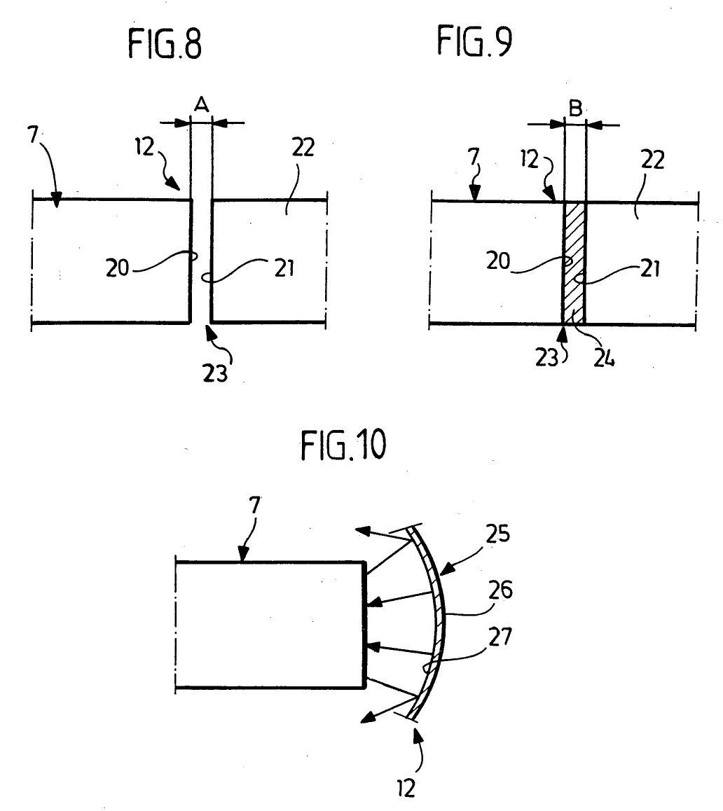

In the case of the exemplary embodiment of FIG. 8, the ground-flat end 20 of the glass fiber 7 is opposite a likewise ground-flat end 21 of a piece of glass fiber 22, a very narrow gap 23 being produced between the ends 20 and 21; the gap width A may for example be of the order of magnitude of 50 mm. This arrangement forms a Fabry-Pérot interferometer and reflects radiation of a quite specific wavelength, which is dependent on the gap width A. If the two ends 20 and 21 are shifted in relation to each other, a shift of the wavelength of the reflected radiation thus also occurs, and this can be detected very sensitively. It is also readily possible in this way to detect for example strains of the implant, which are transferred to the glass fiber 7 and the piece of glass fiber 22.

In the case of the exemplary embodiment of FIG. 9, a similar arrangement is chosen, but an active layer 24 which changes its dimension, for example its volume, in dependence on environmental influences is inserted into the gap 23. This layer may be, for example, a porous structure which swells when liquid enters the pores. The gap width B changes as a result, and this leads to changing of the wavelength of the radiation reflected at the Fabry-Pérot arrangement.

The Fabry-Pérot arrangements of FIGS. 8 and 9 consequently form a sensor member 12 which is connected to the measuring device 11 via the glass fiber 7; in the case of the exemplary embodiments of FIGS. 5 to 7, on the other hand, the glass fiber 7 itself is a sensor element, so this is a case of glass fibers that are themselves sensor fibers.

In the case of the exemplary embodiment of FIG. 10, the glass fiber 7 has an associated sensor member 12 in the form of a pressure sensor 25. This comprises a flexible membrane 26, which is provided on one side with a reflective layer 27. If this pressure sensor 25 is disposed at the end of a glass fiber 7, the electromagnetic radiation reflected back into the glass fiber 7 changes with the deformation of the membrane 26, which takes place pressure-dependently, and consequently a measure of the pressure at the end of the glass fiber 7 is again obtained.

In the case of the exemplary embodiment of FIGS. 1 and 2, glass fibers 7 which are led out from the implant 1 are connected directly or indirectly to the measuring device 11.

This is carried out in a similar way in the case of the embodiment according to FIG. 3, which is set up in a way similar to that of FIG. 1 and in which identical parts are designated by corresponding reference numerals; the connection of the transmission element 8 to the measuring device 11 is symbolized in the case of the exemplary embodiment in FIG. 3 by a line 10, which may be a physical line or a transmission link without a line.

Additionally provided in the case of this embodiment is a radiation source 29, which is connected to one or more glass fibers 30, which are embedded in the synthetic material 6 of the implant 1. In the exemplary embodiment of FIG. 3, only one such glass fiber 30 is represented, connected directly to the radiation source 29; this is to be considered only as a schematic representation. It is also possible here to provide a number of glass fibers 30 which, in a way similar to how the glass fibers 7 are connected to the measuring device, are connected for their part to the radiation source 29, that is to say via transmission elements which could be disposed in the body or outside it, etc.

The radiation source 29 can feed into the glass fibers 30 an electromagnetic radiation which emerges in the interior of the implant 1, where it produces a direct influence on the environment, for example heating-up of the surrounding synthetic material 6 or else additional hardening by increased polymerization or else dissolution of polymerization bonds, etc. Many effects are conceivable here, dependent on the nature of the synthetic material 6 used and on the nature of the electromagnetic radiation fed in. In any event, this fed-in electromagnetic radiation has the effect of influencing the physical data of the synthetic material 6 and possibly of the environment of the implant 1; for example, the strength of the implant can be increased or reduced locally or over its surface area. The location where the effect occurs can be determined by corresponding arrangement of the glass fibers 30 in the implant 1; the type of effect can be determined by corresponding selection of a specific radiation.

The radiation source 29 may be activated completely independently of the measuring device 13; however, it is particularly advantageous if, as represented in FIG. 3, the radiation source 29 has an associated controller 31, which switches the radiation source 29 on and off in dependence on the measured data of the measuring device 11. For this purpose, the measuring device 11 is connected to the controller 31 via a line 28.

If, for example, the measuring device 11 detects that the strain of the implant 1 decreases in a specific region, this is an indication that part of the force transfer has been taken over by healing bone fragments; the strength of the implant 1 can then be reduced by dissolving part of the synthetic material 6 by feeding in electromagnetic radiation in glass fibers 30, with the result that the supporting function of the implant 1 is reduced in a way corresponding to the increase in the stability of the bone connection. Consequently, optimum adaptation of these parameters to each other is possible; it is also beneficial for the healing if the bone connection is increasingly subjected to loading as the healing process proceeds.

In the case of the exemplary embodiment of FIG. 3, the introduction of the radiation generated by the radiation source 29 takes place via glass fibers 30, which are different from the glass fibers 7 of the measuring device.

It is also possible to perform both the measurement of the data relating to the physical state and the feeding-in of electromagnetic radiation via the same glass fibers 7; this is schematically represented in FIG. 4. For this purpose, an optical switch 33, which selectively permits a connection of the glass fibers 7 to the measuring device 11 or the radiation source 29, is connected between the transmission element 8 on the one hand and the measuring device 11 and the radiation source 29 on the other hand. This is symbolically indicated in FIG. 4 by the double-headed arrow C. Switches of this type are available in various ways; they may be mechanical switches, which for example displace a glass fiber between two coupling-in points, or else switches which operate electromagnetically, piezoelectrically or thermally; a large number of different switches that can be used for this purpose are known here to a person skilled in the art.

The optical switch 33 may optionally also be automatically actuated, ensuring as a result that for example the glass fiber 7 is used alternately for performing a measurement of the physical state and for feeding in radiation energy for influencing the environment of the glass fiber.

What is claimed:

1. Medical implant system with an implant made of a composite material in which glass fibers are embedded, a sensor element which is embedded in the implant and comprises at least one of the glass fibers being coupled to a measuring device which determines a physical property of the sensor element or its environment or changing of this property, wherein the glass fibers are embedded in the composite material as mechanical reinforcement in the form of a woven fabric, a knitted fabric or a fleece.

2. Implant system according to claim 1, wherein the glass fibers are distributed in the composite material over the entire extent of the implant.

3. Implant system according to claim 1, wherein the measuring device feeds electromagnetic radiation into the sensor element and determines physical properties of the sensor element or of its environment from the type of radiation that passes through and/or is reflected.

4. Implant system according to claim 3, wherein the glass fiber of the sensor element is provided with a radiation-reflecting coating.

5. Implant system according to claim 1, wherein the sensor element substantially consists of the glass fiber forming a sensor fiber.

6. Implant system according to claim 5, wherein at least one region acting as a Bragg grating is incorporated in the sensor fiber.

7. Implant system according to claim 5, wherein a substance that is induced to fluoresce by the fed-in electromagnetic radiation and the fluorescent properties of which undergo changes under the effect of the chemical environment outside the sensor fiber is embedded in the sensor fiber.

8. Implant system according to claim 4, wherein the radiation-reflecting coating consists of a substance which changes the reflection behavior for the electromagnetic radiation in the sensor fiber under the effect of the chemical environment outside the sensor fiber.

9. Implant system according to claim 1, wherein the sensor element comprises the glass fiber and a further sensor member, which is coupled to the measuring device via the glass fiber.

10. Implant system according to claim 9, wherein the sensor member is a pressure sensor with a flexible membrane and a mirror element which can be moved by the latter and reflects the electromagnetic radiation fed into the glass fiber differently according to position.

11. Implant system according to claim 9, wherein the sensor member is a Fabry-Pérot interferometer.

12. Implant system according to claim 11, wherein in the Fabry-Pérot interferometer is formed as a thin-film interferometer that is brought into contact with the end of the glass fiber and the active film of which undergoes dimensional changes under the influence of the environment.

13. Implant system according to claim 11, wherein the Fabry-Pérot interferometer comprises two glass fibers with polished end faces, the spacing (B) between which can be changed by environmental influences.

14. Implant system according to claim 1, wherein the glass fiber of the sensor element is connected directly to the measuring device.

15. Implant system according to claim 14, wherein the measuring device is a microcontroller that is capable of being implanted in the body.

16. Implant system according to claim 1, wherein the glass fiber is connected to a transducer, which exchanges signals with the measuring device without a physical connection.

17. Implant system according to claim 16, wherein the transducer is capable of being implanted in the body.

18. Implant system according to claim 16, wherein the transformer is a transducer.

19. Implant system according to claim 16, wherein the transducer is a light source which has an associated light receiver.

20. Implant system according to claim 19, wherein the light source emits electromagnetic radiation in the range between 650 and 1000 nm.

21. Implant system according to claim 1, wherein the measuring device has an associated radiation transmitter, which transports radiation into the interior of the implant via a glass fiber in the implant.

22. Implant system according to claim 21, wherein the transport of the radiation takes place via the glass fiber of a sensor element.

23. Implant system according to claim 21, wherein the transport of the radiation takes place via a glass fiber which is embedded in the implant in addition to the glass fiber of a sensor element.

24. Implant system according to claim 21, wherein the wavelength and intensity of the transported radiation are chosen such that the radiation induces mechanical and/or material changes in the composite material of the implant.

25. Implant system according to claim 21, wherein the measuring device and the radiation transmitter have an associated controller, which activates the radiation transmitter in dependence on the measured variables of the measuring device.
